(19) United States
(12) Patent Application Publication
(10) Pub. No.: US 2011/0288525 A1
(43) Pub. Date: Nov. 24, 2011

(54) OCULAR SURGICAL PROCEDURE

(76) Inventors: Paul Hallen, Colleyville, TX (US); Timothy Murray, Coral Gables, FL (US)

(21) Appl. No.: 13/111,478

(22) Filed: May 19, 2011

Related U.S. Application Data
(60) Provisional application No. 61/346,672, filed on May 20, 2010.

(51) Int. Cl. A61F 9/00

(52) U.S. Cl. 604/506

(57) ABSTRACT

Aspects of forming a wound-conforming pressure seal in the eye, such as upon completion of an ocular surgical procedure, are described herein. Forming the seal includes introducing a plug member into an opening formed in the eye to block vitreous from exiting from the eye through the opening. Consequently, the seal is operable to substantially reduce or eliminate hypotony (i.e., lowering of intraocular pressure below safe levels) and/or endophthalmitis (i.e., infections to the eye).

### CROSS-REFERENCE TO RELATED APPLICATIONS

This application claims the benefit of U.S. Provisional Patent Application No. 61/346,672, filed May 20, 2010, which is incorporated by reference in its entirety.

### TECHNICAL FIELD

The present disclosure relates to various aspects of gaining access to an interior of the eye. Particularly, the present disclosure describes features associated with gaining entry into an interior of the eye and closing the eye once access to the eye's interior is no longer required and/or desired.

### BACKGROUND

In many surgical procedures involving the eye, an opening may be formed in the sclera of the eye to permit access to an interior of the eye. For example, in some instances, the opening may be formed in the sclera of the eye to permit access to an interior volume of the posterior segment of the eye. The opening may be utilized, for example, to pass materials and/or instruments into and/or out of the eye, such as to perform a medical procedure therein, or otherwise gain access to interior structures of the eye.

### SUMMARY

The present disclosure relates to surgical procedures related to the eye. According to one aspect, the disclosure describes a method for sealing an opening formed in the eye, the opening having a cannula disposed therein. A plug member may be inserted into a passageway formed in the cannula disposed in the opening of the eye, and at least a portion of the plug member may be positioned at a location within the passageway adjacent to where the eye engages the cannula at the opening. Additionally, the cannula may be retracted from the opening such that at least a portion of the plug member resides in the opening to form a seal.

Another aspect encompasses a method of forming a wound-conforming seal during one of a 20 gauge, 23 gauge, 25 gauge, or 27 gauge ocular surgical procedure. The method may include coupling a collagen plug to a distal end of a surgical instrument. The distal end may be introduced into an interior passageway of a cannula extending through an opening in the sclera of an eye, and the collagen plug may be positioned within the interior passageway via the surgical instrument such that at least a portion of the collagen plug is within the opening. The cannula may be removed from the opening formed in the sclera such that at least a portion of the collagen plug resides within the opening formed in the sclera to seal the opening. The collagen plug may be released from the surgical instrument.

A further aspect relates to an ocular surgical procedure. The ocular surgical procedure may include forming an opening in the sclera of the eye with a trocar. A cannula may be inserted into the opening formed in the eye. The cannula may include an interior passageway providing communication between an interior of the eye and an exterior of the eye. A collagen plug may be coupled to a surgical instrument, and the collagen plug may be inserted into the interior passageway with the surgical instrument. The collagen plug may be positioned within the interior passageway to a position such that at least a portion of the collagen plug is disposed within the opening formed in the sclera, and the cannula may be removed from the opening while the position of the collagen plug relative to the opening formed in the sclera may be substantially maintained such that at least a portion of the collagen plug resides within the opening formed in the sclera when the cannula is removed.

The various aspects may include one or more of the following features. The plug member may elute medication to treat a condition of the eye. The eluted medication may include an anti-inflammatory medication. The eluted medication may include anti-VEGF medication. The eluted medication may be an anti-infective medication. Inserting a plug member into a passageway formed in a cannula disposed in an opening formed in the eye may include coupling the plug member to a first end of an instrument and introducing the first end of the instrument coupled to the plug member into the passageway. Also, positioning at least a portion of the plug member at a location within the passageway to where the eye engages the cannula at the opening may include extending the instrument through the passageway along a longitudinal axis thereof until at least a portion of the plug member is located within the passageway at a location where the opening annularly surrounds the cannula. Retracting the cannula from the opening such that at least a portion of the plug member resides in the opening to form a seal may include releasing the plug member from the instrument and withdrawing the instrument. Also, the plug member may be formed from collagen. Further, the plug member may be infused with medication. The plug member may include at least one flange portion.

The various aspects may also include one or more of the following features. Medication may be introduced into the collagen plug. The medication may be one of an anti-inflammatory medication, an anti-infective medication, or an anti-VEGF medication. The collagen plug residing in the opening formed in the eye may be hydrated with a fluid drug. The surgical procedure may be one of a 20 gauge, 23, gauge, 25 gauge, or 27 gauge surgical procedure. The surgical instrument for inserting the collagen plug into the interior passageway may be a trocar. Medication may be introduced into the collagen plug. Further, the collagen plug may be adapted to elute medication introduced therein. The medication introduced into the collagen plug may include at least one of an anti-inflammatory medication, an anti-infective medication, or an anti-VEGF medication. The collagen plug residing in the opening formed in the eye may be formed in the sclera with a fluid drug. The collagen plug may include at least one flange portion. Medication may be eluted into the eye from the collagen plug.

The details of one or more implementations of the present disclosure are set forth in the accompanying drawings and the description below. Other features, objects, and advantages will be apparent from the description and drawings, and from the claims.

### DESCRIPTION OF DRAWINGS

FIG. 1 is a partial cross-sectional view of a portion of the eye having an opening formed therein and a cannula inserted through the opening to provide communication between an interior and exterior of the eye.

FIGS. 2-5 show installation of a plug member into an opening formed in the eye.

FIGS. 6-8 show cross-sectional views of additional example plug members.

FIGS. 9-11 show cross-sectional views of further example plug members.

### DETAILED DISCLOSURE

Access to an interior of the eye, such as to an interior of the posterior segment of the eye, may be required during a surgical procedures. For example, access to the interior of the posterior segment may be required when access to the retina is needed. Access may be gained by forming an opening in the sclera of the eye. In some instances, the opening may be formed with a trocar. Additionally, the trocar may be utilized to insert a cannula in the opening of the sclera

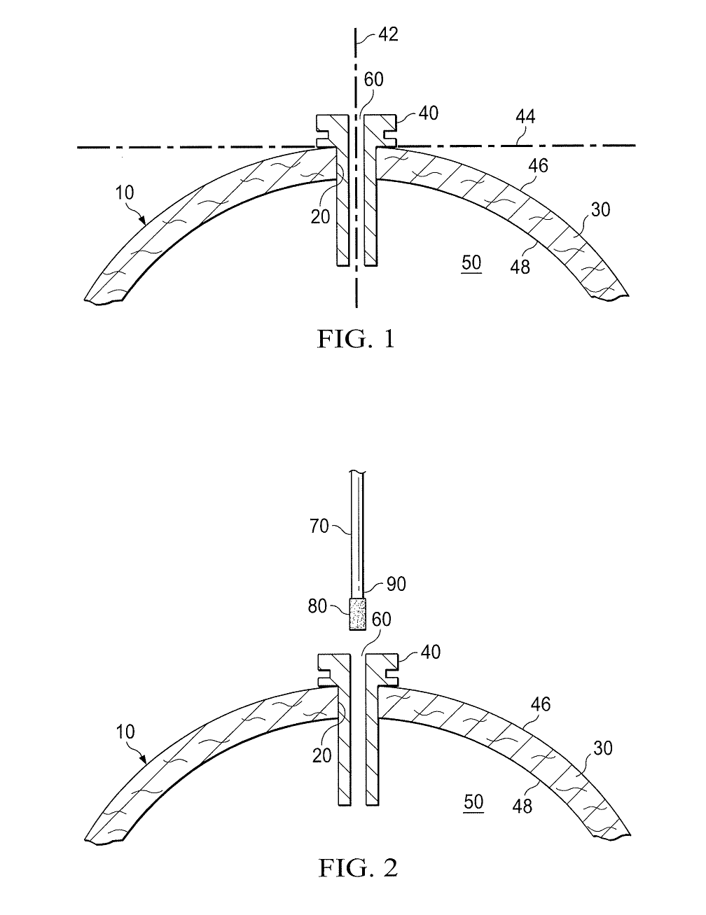

FIG. 1 shows a partial cross-sectional view of an eye 10 in which an opening 20 (also referred to as a wound) is formed in the sclera 30. A cannula 40 is shown disposed in the opening 20. The cannula 40 may be used to aspirate materials from the interior 50 of the eye 10 and/or introduce materials or equipment, such as vitrectomy surgical devices, into the interior 50 of the eye 10. Further, more than one cannula may be introduced into other openings formed in the sclera. Thus, during some procedures, several openings, having a cannula disposed respectively therein, may be formed in the sclera. Although the cannula 40 is shown in FIG. 1 disposed in the opening 20 such that a longitudinal axis 42 of the cannula is oriented perpendicularly or substantially perpendicularly to a plane 44 tangent to an outer surface 46 of the eye 10, the cannula 40 may be disposed at any desired or suitable orientation. That is, the cannula 40 may be inserted through the sclera and form any desired or suitable angle between the longitudinal axis 42 and plane 44.

The cannula 40 defines an interior passageway 60 through which one or more instruments may be repeatedly inserted and/or retracted during a surgical procedure, for example. Further, the cannula 40 provides repeated access to the interior 50 of the eye 10 without the need for the medical practitioner to relocate the opening 20 formed in the sclera 30 and without causing repeated trauma to the eye structures as the instruments and/or materials are inserted into and/or withdrawn from the eye 10 through the opening 20.

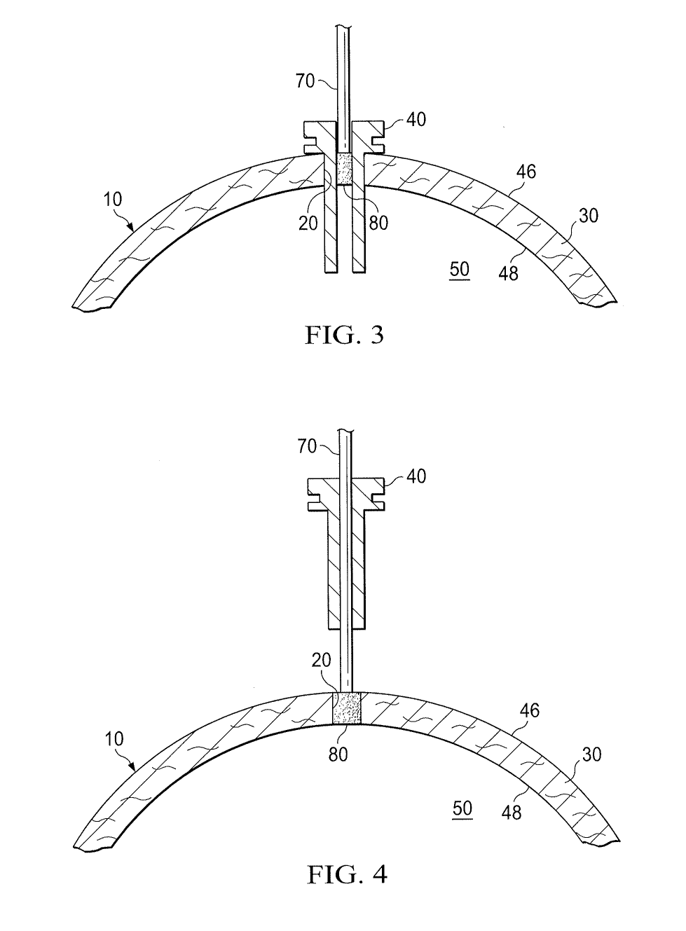

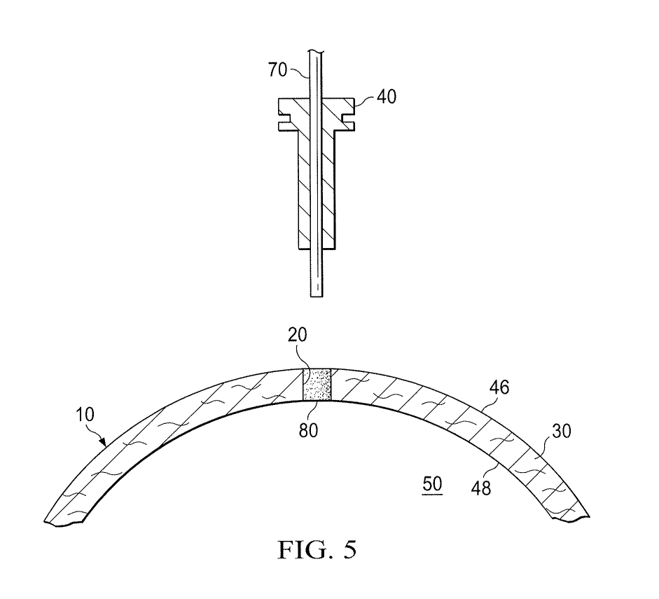

The opening 20 formed in the sclera 30 may be closed, such as at the conclusion of a medical procedure. FIGS. 2-5 illustrate insertion of a plug member into an opening formed in the sclera of an eye. An instrument 70, such as a trocar, having a plug member 80 disposed at a first end 90 of the instrument 70 may be inserted into the passageway 60 of the cannula 40, as shown in FIGS. 2-3. The plug member 80 may be disposed within the passageway 60 of the cannula 40 such that at least a portion of the plug member 80 is positioned within the opening 20. That is, the plug member 80 may be located within the passageway 60 at a location adjacent where the cannula 40 engages the sclera 30. That is, in some implementations, the plug member 80 may be longitudinally positioned along the passageway 60 such that at least a portion of the plug member 80 is positioned between the outer surface 46 and an inner surface 48 of the eye. The cannula 40 may be retracted along the instrument 70 as the plug member 80 is maintained or substantially maintained at its position relative to the opening 20, as shown in FIG. 4. In some implementations, the cannula 40 may be removed with forceps. The plug member 80 may be released from the instrument 70 and the instrument 70 retracted, as shown in FIG. 5. As a result, the plug member 80 may be inserted into the opening 20. The plug member 80 may be hydrated by aqueous humor, i.e., fluid naturally generated by the ciliary process of the eye, or other fluid, such as a fluid drug applied directly or indirectly to the plug member 80. Consequently, the plug member 80 forms a wound-conforming pressure seal operable to provide one or more of the benefits described herein.

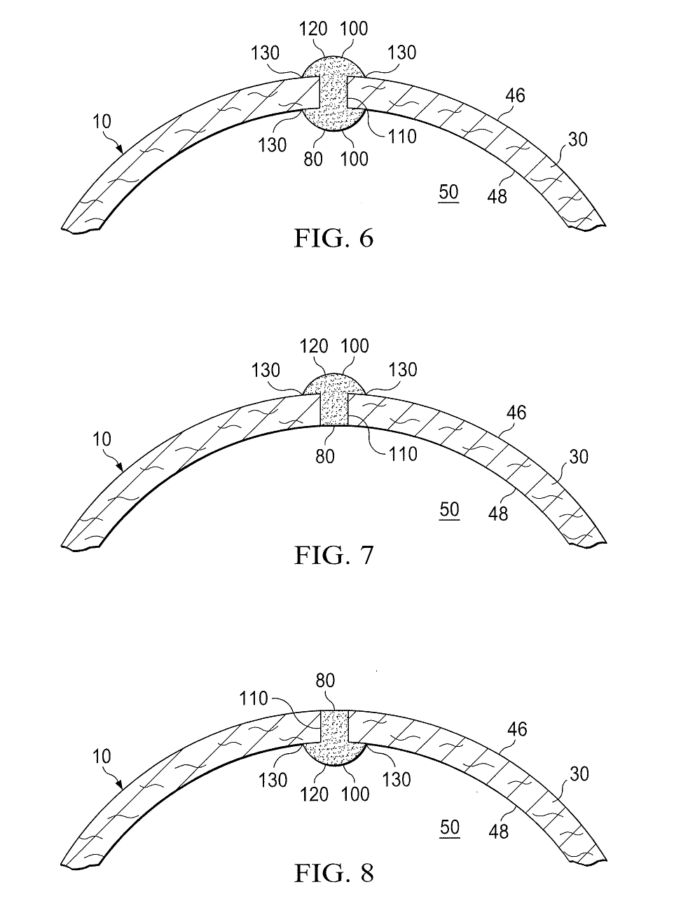

Although FIGS. 2-5 illustrate the plug member 80 as having a substantially cylindrical shape, the plug member 80 may have other shapes. For example, the plug member 80 may have one or more flanged portions, as shown in FIGS. 6-8. The example plug member 80 shown in FIG. 6 has flanged portions disposed at opposing ends of a cylindrical portion 110.

In other implementations, as shown in FIG. 7, the plug member 80 may include a flange portion 100 disposed at a single end of the cylindrical portion 110. The plug member 80 having a single flange portion 100 may be inserted into the eye 10 having a first orientation (shown in FIG. 7) in which the flange portion is disposed at the exterior of the eye or a second orientation (shown in FIG. 8) in which the flange portion 100 is disposed in the interior 50 of the eye 10. The flange portion(s) 100 may be compressible to aid in insertion into and/or retention within the eye 10. Thus, upon removal of the cannula 40 when the plug member 80 is in position within the opening 20, the flange portion(s) 100 expand to aid in holding the plug member 80 in position.

As shown in FIGS. 6-8, the flange portions 100 may have a surface 120 that defines a tapered edge 130. In some instances, the surface 120 may be a curved surface as shown in FIGS. 6-8. The surface 120 and/or the tapered edge 130 may facilitate retention of the plug member 80 within the opening 20. For example, the curved surface 120 and/or tapered edge 130 may prevent a tissue contacting the eye from inadvertently dislodging the plug member 80, such as by relative movement between the eye 10 and the adjacent, contacting tissue.

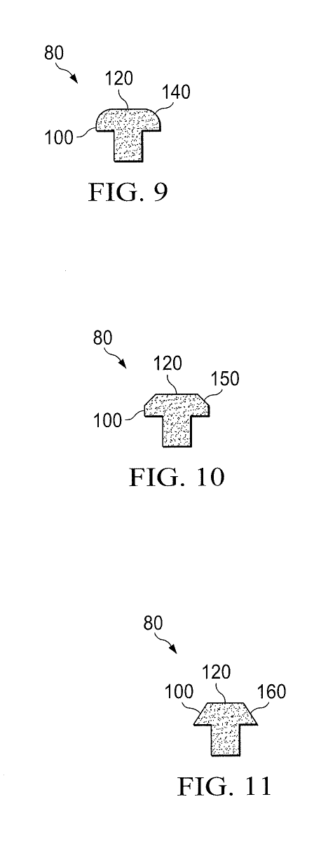

Further, although the surface 120 of the example plug member 80 is shown as having a curved shape, the disclosure is not so limiting. Rather, in other implementations, the surface 120 may be flat with or without a radiused edge 140 (FIG. 9) or a chamfered edge 150 (FIG. 10). Another example plug member 80 shown in FIG. 11 may have a tapered surface 160. In still other implementations, the surface 120 may form define a semi-hemispherical shape. However, the surface 120 may have any suitable or desired shape. Further, although FIGS. 9-11 show a plug member 80 having a single flange portion 110, in other instances, the plug member 80 may include two flange portions 110 that may have an identical shape or a different shape than the other flange portion 110.

For example, use of a plug member 80 having the second orientation may be beneficial in maintaining the plug member 80 within the opening 20 as the cannula 40 is withdrawn from the eye 10. When the plug member 80 is at a desired location within the opening 20, as the cannula 40 is withdrawn past the flange portion 100, the flange portion 100 expands to seal the opening 20. Further, expansion of the flange portion 100 within the eye 10 provides mechanical resistance to withdrawal of the plug member 80 as the cannula 40 is further withdrawn from the eye 10. Thus, the flange portion 100 not only aids in sealing the opening 20, the flange portion 100 also provides a locating function to maintain a position of the plug member 80 as the cannula 40 is withdrawn from the eye 10.

While the flange portion(s) 100 of the plug member 80 is described as being compressible (and expandable to retain its original shape after a compressive force is removed), other portions of the plug member 80 may also be compressible to some extent. In some instances, the cylindrical portion 110 may be sized to substantially correspond to the interior passageway 60 of the cannula 40. That is, in some instances, the cylindrical portion 110 may have a dimension (e.g., diameter) that corresponds to the dimension (e.g., diameter) of the interior passageway 60. In other instances, the cylindrical portion 110 may be slightly larger than the interior passageway 60 such that, when inserted into the interior passageway 60, the cylindrical portion 110 is compressed slightly. In still other instances, the cylindrical portion 110 may be slightly smaller than the interior passageway 60 (e.g., the diameter of the cylindrical portion 110 may be slightly smaller than a diameter of the interior passageway 60).

In some implementations, the plug member 80 may be a collagen plug. In some implementations, the plug member 80 may be adapted to absorb and elute one or more materials, such as one or more medications. For example, the plug member 80 may be adapted to absorb and elute an anti-inflammatory material. Example anti-inflammatory materials may include triamcinolone acetonide as well any other suitable material and/or medication for treating and/or reducing inflammation of ocular tissues. Thus, according to some implementations, the plug member, such as plug member 80, may be operable to decrease swelling of the eye, particularly at the site of the opening, such as opening 20. In still other implementations, the plug member 80 may be adapted to absorb and elute anti-vascular endothelial growth factor (“anti-VEGF”) materials. Example anti-VEGF materials may include Lucentis®, produced by Genetech, Inc. of 1 DNA Way, South San Francisco, Calif. 94080. In still other implementations, the plug member 80 may be adapted to absorb and elute an anti-infective material. An example anti-infective material may include Vigamox® (moxiflocacin) produced by Alcon Laboratories, Inc. of South Freeway, Fort Worth, Tex. 76134. Another example anti-VEGF material may include Avastin®, also produced by Genetech, Inc. Consequently, in some instances, the plug member, such as plug member 80, may be adapted to reduce or eliminate the growth of abnormal blood vessels in at least a portion of the eye.

Still further, in some instances, a plurality of drugs may be absorbed into the plug member 80. Thus, a combination of different medications that may include, for example, one or more anti-inflammatory medications, anti-VEGF medications, anti-infective medications, and/or other medications may be may be absorbed into the plug member 80. Still other types of medications and combinations thereof other than or in combination with one or more of the materials described above may be used. Further, other types of biological agents and/or small molecule medicaments may be included in the plug member 80 for elution into the eye 10. As such, when inserted into the eye 10, the plug member 80 is operable to elute multiple, different medications into the eye 10. In still other implementations, the plug member 80 may be used as a port for the injection of one or more of the medications and/or substances described herein. As such, one or more materials may be directly injected into the eye through the plug member 80.

One or more of the implementations described herein may be used in combination with 20 gauge (“20 g”) to 27 gauge (“27 g”) wounds to the eye or 20 g to 27 g surgical techniques. For example, one or more of the implementations described herein may be used in combination with 20 g, 23 gauge (“23 g”), 25 gauge (“25 g”), or 27 g surgical procedures. However, the scope of use is not so limited, and one or more of the implementations described above may be use with larger or smaller openings or with larger or smaller surgical techniques. A plug member, such as plug member 80, may form a pressure seal to seal the opening formed in the sclera, such as opening 20, while the eye heals. As a result, hypotony (i.e., the reduction of intraocular pressure to unsafe levels) may be substantially reduced or eliminated. Additionally, the plug member 80 may substantially reduce or prevent intraocular vitreous strands from wicking out onto external conjunctiva and, thereby, proving a pathway for microbes to enter the eye and cause infections. These infections, referred to as endophthalmitis, can severely damage the eye and cause blindness.

In still other implementations, a plug member, such as one or more of the plug members 80 described above, may be inserted into a wound formed in the eye without the use of a cannula. For example, a plug member may be inserted into a cataract surgical entry wound or other ocular surgical wound without the use of a cannula. Thus, a plug member may be inserted into a wound formed in the eye with or without the use of a cannula.

Further, it should be understood that, although many aspects have been described herein, some implementations may include all of the features, while others may include some features while omitting others. That is, various implementations may include one, some, or all of the features described herein.

A number of implementations have been described. Nevertheless, it will be understood that various modifications may be made without departing from the spirit and scope of the disclosure. Accordingly, other implementations are within the scope of the following claims.

What is claimed is:

1. A method for sealing an opening formed an eye, the opening having a cannula disposed therein, the method comprising:
inserting a plug member into a passageway formed in the cannula disposed in the opening of the eye;
positioning at least a portion of the plug member at a location within the passageway adjacent to where the eye engages the cannula at the opening; and
retracting the cannula from the opening such that at least a portion of the plug member resides in the opening to form a seal.

2. The method of claim 1 further comprising eluting medication from the plug member to treat a condition of the eye.

3. The method of claim 2, wherein eluting medication from the plug member to treat a condition of the eye comprises eluting anti-inflammation medication from the plug member.

4. The method of claim 2, wherein eluting medication from the plug member to treat a condition of the eye comprises eluting anti-VEFG medication from the plug member.

5. The method of claim 2, wherein eluting medication from the plug member to treat a condition of the eye comprises eluting an anti-infective medication from the plug member.

6. The method of claim 1, wherein inserting a plug member into a passageway formed in the cannula disposed in the opening of the eye comprises:
coupling the plug member to a first end of an instrument; and
introducing the first end of the instrument coupled to the plug member into the passageway.

7. The method of claim 1, wherein positioning at least a portion of the plug member at a location within the passageway adjacent to where the eye engages the cannula at the opening comprises extending the instrument through the passageway along a longitudinal axis thereof until at least a portion of the plug member is located within the passageway at a location where the opening annularly surrounds the cannula.

8. The method of claim 7, further comprising releasing the plug member from the instrument and withdrawing the instrument.

9. The method of claim 1, wherein the plug member is formed from collagen.

10. The method of claim 1, wherein the plug member comprises at least one flange portion.

11. The method of claim 1 further comprising infusing medication into the plug member.

12. A method of forming a wound-conforming seal during one of a 20 gauge, 23 gauge, 25 gauge, or 27 gauge ocular surgical procedure, the method comprising:
coupling a collagen plug to a distal end of a surgical instrument;
introducing the distal end into an interior passageway of a cannula extending through an opening in the sclera of an eye;
positioning the collagen plug within the interior passageway via the surgical instrument such that at least a portion of the collagen is within the opening;
removing the cannula from the opening formed in the sclera such that at least a portion of the collagen plug resides within the opening formed in the sclera to seal the opening; and
releasing the collagen plug from the surgical instrument.

13. The method of claim 12 further comprising introducing medication into the collagen plug.

14. The method of claim 13, wherein the medication is one of an anti-inflammatory medication, an anti-infective medication, or an anti-VEGF medication.

15. The method of claim 12 further comprising hydrating the collagen plug disposed in the opening formed in the eye with a fluid drug.

16. The method of claim 12, wherein the collagen plug comprises at least one flange portion.

17. An ocular surgical procedure comprising:
forming an opening in the sclera of the eye with a trocar;
inserting a cannula into the opening formed in the eye, the cannula comprising an interior passageway providing communication between an interior of the eye and an exterior of the eye;
coupling a collagen plug to a surgical instrument;
inserting the collagen plug into the interior passageway with the surgical instrument;
positioning the collagen plug within the interior passageway to a position such that at least a portion of the collagen plug is disposed within the opening formed in the sclera; and
removing the cannula from the opening while substantially maintaining the position of the collagen plug relative to the opening formed in the sclera such that at least a portion of the collagen plug resides within the opening formed in the sclera when the cannula is removed.

18. The ocular surgical procedure of claim 17, wherein the scale of the surgical procedure is one of a 20 gauge, 23 gauge, 25 gauge, or 27 gauge surgical procedure.

19. The ocular surgical procedure of claim 17, wherein the surgical instrument is a trocar.

20. The ocular surgical procedure of claim 17 further comprising introducing medication into the collagen plug.

21. The ocular surgical procedure of claim 20, further comprising eluting the medication from the collagen plug into the eye.

22. The ocular surgical procedure of claim 20, wherein introducing medication into the collagen plug comprises introducing at least one of an anti-inflammatory medication, an anti-infective medication, or an anti-VEGF medication.

23. The ocular surgical procedure of claim 17 further comprising hydrating the collagen plug residing in the opening formed in the sclera with a fluid drug.
